(19) United States
(12) Patent Application Publication
(10) Pub. No.: US 2022/0080100 A1
(43) Pub. Date: Mar. 17, 2022

(54) URINARY CATHETER ASSEMBLY

(71) Applicants: Michael D. Jones, Pacific Grove, CA (US); Nicole E. Tonti, Santa Cruz, CA (US)

(72) Inventors: Michael D. Jones, Pacific Grove, CA (US); Nicole E. Tonti, Santa Cruz, CA (US)

(21) Appl. No.: 17/385,587

(22) Filed: Jul. 26, 2021

Related U.S. Application Data
(60) Provisional application No. 63/077,867, filed on Sep. 14, 2020.

(51) Int. Cl. A61M 1/00

(52) U.S. Cl. CPC A61M 1/86; A61M 2205/3337; A61M 2210/1096; A61M 1/69

(57) ABSTRACT

A condom-catheter system includes a condom-catheter with a tube extending to a collection bag. A rotary junction is disposed between the tube that is connected to the condom-catheter and the tube that is connected to the collection bag.

### PRIORITY APPLICATION

This application claims the benefit of U.S. Provisional Patent Application No. 63/077,867, filed 14 Sep. 2020; which application is incorporated herein by reference.

### BACKGROUND

A condom-catheter is the suggested catheter option, by urologists, for many patients. FIG. 1 shows the components of a condom-catheter and catheter drainage bag assembly. The two common drainage bags are a leg-strapped bag, and a free-hanging bag.

The free-hanging drainage bag has two significant advantages. Its larger volume allows for less frequent emptying, and pants need not be removed to empty it. A hose can be routed out through the pant leg, and the bag can be hung on the side of a wheelchair, bed, etc.

Condom-catheters often get twisted. This causes them to leak and fail (followed by the inconvenient replacement process). There are several things that can cause this to happen, but the most common cause of condom-catheter twisting occurs when the drainage bag is inadvertently flipped. Anytime a patient is transferred or has their pants put on, there is a significant risk for the catheter bag to be flipped—causing the catheter to twist, fail, and be replaced.

It is desirable to provide a technology addressing these issues.

### SUMMARY

A catheter drainage bag, coupler assembly is provided to prevent condom-catheter failures caused by twisting of the connected hose. It does this by adding some simple, inexpensive, available technologies.

A solution is provided that includes putting a rotary union, preferably with very low friction, somewhere along the tube so that the twist does not travel all the way up the chain to the client. The rotary union does not interrupt the flow of urine into the bag as it is twisted, and it keeps the twisting movement from going further up.

An embodiment includes locating the rotary union right above the connection between the tube that is connected to the condom-catheter and the tube that is connected to the collection bag.

Also, the tube in some embodiments can include a pressure relief valve for the urine. The rotary union can be placed at or adjacent to a junction in the tube at the pressure relief valve.

The junction area including the pressure relief valve has a tendency to catch on the client's leg or other objects as the client is moved. This pressure relief valve sticks out from the rest of the tubing there, so it creates an opportunity to catch things. Embodiments of the technology can include a cover of the pressure relief valve and rotary union junction that smooths the edges and reduces the likelihood that the junction will catch and snag.

The cover can comprise a cylindrical tube that slips over the junction, and can have an opening aligned with the pressure relief valve. It can comprise medical grade plastic. It can be preferred that the covering has a low friction surface. This reduces the number of edges on which the combined pressure relief valve and rotary union junction can snag. Also, the covering can be contoured so as to reduce or eliminate sharper edges.

The technologies described herein can be applied in combination, and individually, to an external urinary catheter. It can be applied in other types of catheters as well, which may have a drain tube extending externally to a collection bag. It can be applied to assemblies in which there is a bag attached to a catheter or tube where there may be a twist or turn of the bag, or a flipping of the bag.

### BRIEF DESCRIPTION OF FIGURES

FIG. 1A to 1C are perspective views of embodiments of a urinary condom-catheter system.

FIG. 2 is a simplified diagram of a rotary union suitable for the system of FIG. 1.

FIG. 3 is a simplified diagram of a rotary union like that of FIG. 2, rotated ninety degrees.

### DETAILED DESCRIPTION

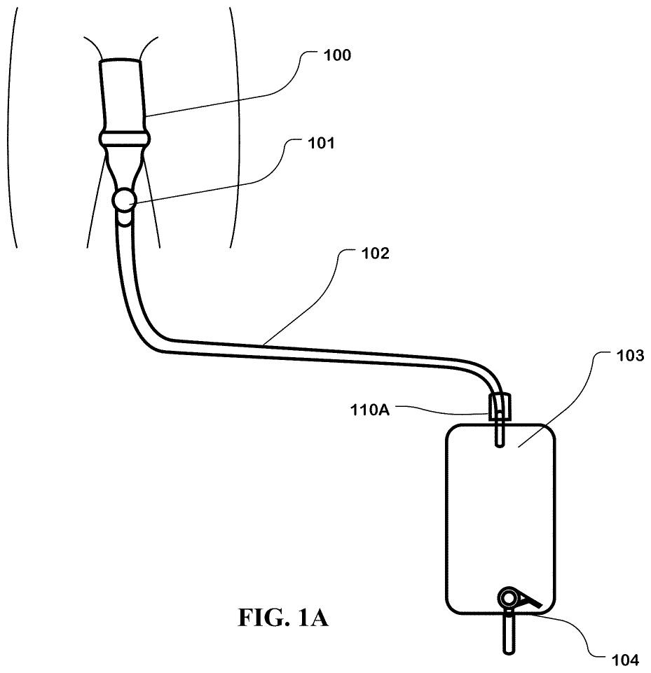

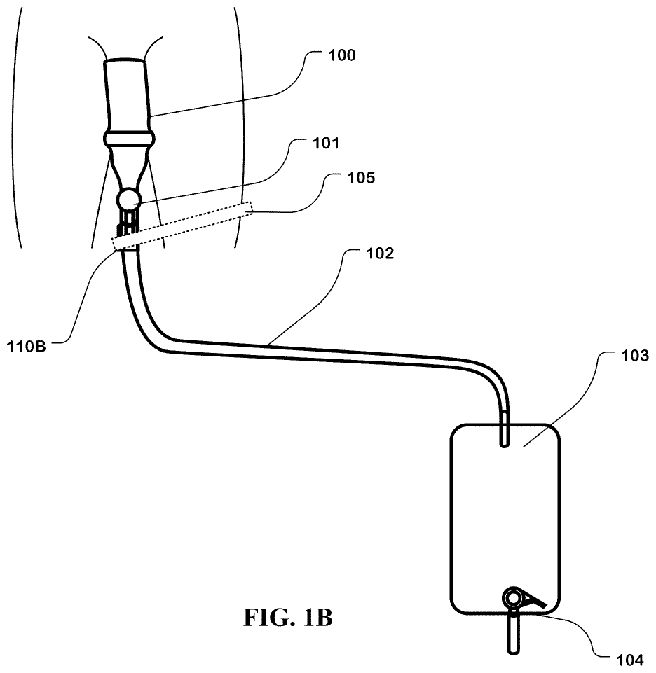

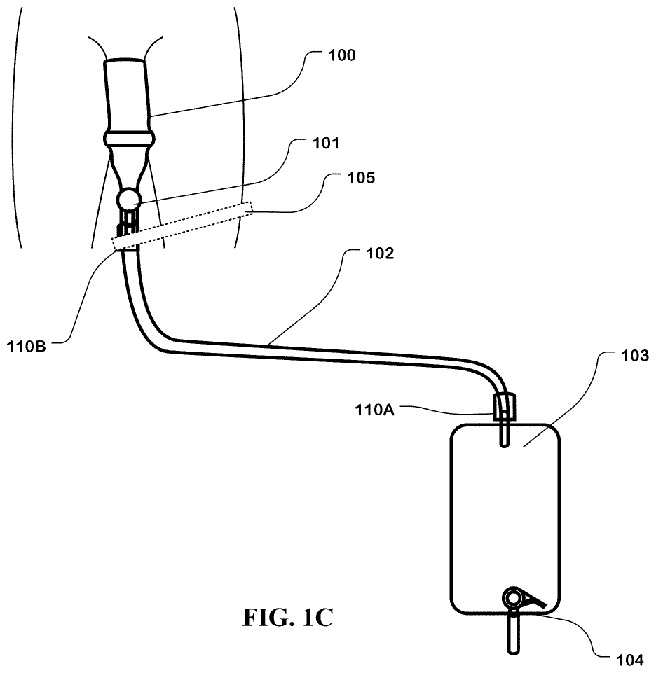

A detailed description of a condom-catheter system is described with reference to FIGS. 1-3.

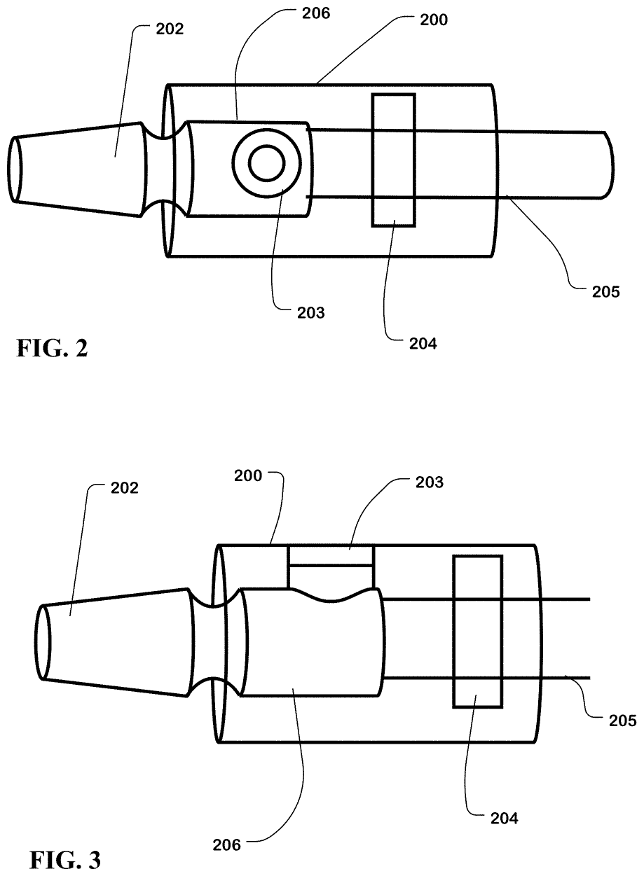

We describe a rotation mechanism assembly (inside or part of the catheter drainage bag coupler assembly). A plastic extrusion (including the pressure relief valve) is made in two mating pieces which are sealed from leaking. The male assembly piece rotates freely (axially) within the female piece. This is physically possible as the rotation mechanism and mating seal are both of sufficiently low friction. FIG. 2 illustrates examples of the functional components.

In some cases, there is a leg-strap that acts as an inertial anchor for the female half of the coupler assembly. The strap fastens with Velcro in some cases, enabling easy adjustments.

FIG. 1A illustrates a condom-catheter system having a condom-catheter 100 secured to a patient. A condom-catheter coupler 101 is connected to the condom-catheter 100 and directs fluid into a tube 102. The tube 102 extends to a drainage bag 103, which has an outlet valve 104. A rotary junction assembly 110A is secured to the tube 102. In this embodiment, the rotary junction assembly 110 is secured adjacent the drainage bag 103, distal to the condom-catheter coupler 101. When the patient turns or drainage bag 103 is inadvertently flipped during a patient transfer, the rotary junction assembly 110 prevents that twist from being transmitted up to the condom-catheter coupler 101 of the condom-catheter 100.

FIG. 1B illustrates a condom-catheter system having a condom-catheter 100 secured to a patient. A condom-catheter coupler 101 (see also 202 of FIG. 2) is connected to the condom-catheter 100 and directs fluid into a tube 102. The tube 102 extends to a drainage bag 103, which has an outlet valve 104. A rotary junction assembly 110B is secured to the tube 102. In this embodiment, the rotary junction assembly 110B is secured adjacent to, or proximal to, the condom-catheter coupler 101 on the condom-catheter 100. A leg strap 105 connects to the outside of rotary junction assembly 110B, which can be secured to or part of a female extrusion piece of the rotary junction assembly 110B. It also straps around one of the patient's legs at a time. This acts as an inertial anchor, if that is needed. The leg strap 105 can comprise a hook and loop fastener (e.g. Velcro) making it easy to adjust and switch to the other leg when needed. When the drainage bag 103 is inadvertently flipped during a patient transfer, causing the tube 102 to twist, the rotary junction assembly 110B prevents that twist from being transmitted up to the condom-catheter 100.

FIG. 1C illustrates a condom-catheter system having a condom-catheter 100 secured to a patient. A condom-catheter coupler 101 (see also 202 of FIG. 2) is connected to the condom-catheter 100 and directs fluid into a tube 102. The tube 102 extends to a drainage bag 103, which has an outlet valve 104. A rotary junction assembly 110A is secured to the tube 102. In this embodiment, the rotary junction assembly 110A is secured adjacent the drainage bag 103, and rotary junction assembly 110B is secured adjacent to, or proximal to, the condom-catheter coupler 101 on the condom-catheter 100. A leg strap 105 connects to the outside of rotary junction assembly 110B, which can be secured to or part of a female extrusion piece of the rotary junction assembly 110B. It also straps around one of the patient's legs at a time. This acts as an inertial anchor, if that is needed. The leg strap 105 can comprise a hook and loop fastener (e.g. Velcro) making it easy to adjust and switch to the other leg when needed. When the drainage bag 103 is inadvertently flipped during a patient transfer, causing the tube 102 to twist, the rotary junction assembly 110 prevents that twist from being transmitted up to the condom-catheter 100. In this embodiment, a pressure relief valve can be disposed in the rotary junction assembly adjacent the drainage bag 103, and omitted from the rotary junction assembly adjacent to the condom-catheter coupler 101. Alternatively, pressure relief valves can be disposed in both structures or in a different location along the fluid flow path.

FIG. 2 is a top view of a rotary junction assembly, having a cover 200 enclosing the rotary junction and housing a pressure relief valve 203. A leg strap (not shown) can be secured to the cover. The rotary junction assembly includes a nozzle 202 or other tube coupler, for connection to a condom-catheter coupler 101 on the tip of the condom-catheter 100, or to a mating connection on the drainage bag 103. Nozzle 202 is secured to the cover 200, which also houses the pressure relief valve 203, and to a female coupler of rotary junction 204, so that it is free from rotation of tube 205 and drainage bag 103. A tube 205 (which can be tube 102, or connected to tube 102, of FIGS. 1A to 1C) includes a male coupler of the rotary junction 204. The tube 205 extends out of the assembly for connection to the drainage bag, or to the condom-catheter coupler or to another structure along the fluid flow path.

FIG. 3 is a side view of a rotary junction assembly like that of FIG. 2 rotated by 90°, with the same reference numerals applied to the same elements. The rotary junction assembly includes nozzle 202, or other tube, for connection to the condom-catheter coupler 101 of the condom-catheter 100, or to the drainage bag. Nozzle 202 is secured to the cover 200, which also houses the pressure relief valve 203, and to a female coupler of rotary junction 204, so that it is free from rotation of tube 205 and drainage bag 103. A tube 205 (which can be tube 102, or connected to tube 102, of FIGS. 1A to 1C) includes a male coupler of the rotary junction 204. The tube 205 extends out of the assembly for connection to the drainage bag, to the condom-catheter coupler or to another structure along the fluid flow path.

The rotary junction assembly (e.g. FIGS. 2 and 3) includes a cover 200, such as a plastic cover, which tends to prevent snags on the pressure relief valve 203 and the rotary coupler 204, during movement and adjustment of the system. In some embodiments, a leg strap (not shown) can be secured to the cover 200.

While the present invention is disclosed by reference to the preferred embodiments and examples detailed above, it is to be understood that these examples are intended in an illustrative rather than in a limiting sense. It is contemplated that modifications and combinations will readily occur to those skilled in the art, which modifications and combinations will be within the spirit of the invention and the scope of the following claims.

What is claimed is:

1. A urinary catheter system, comprising:
a condom-catheter and a collection bag;
rotary union assembly, including a rotary coupler sealed from leaking and having a female mating piece and a male mating piece that rotates freely within the female mating piece;
a tube extending from the condom-catheter to the rotary union assembly; and
a tube extending from the rotary union assembly to the collection bag.

2. The urinary catheter system of claim 1, the rotary union assembly including a cover over the rotary coupler.

3. The urinary catheter system of claim 1, the rotary union assembly including a pressure relief valve disposed adjacent the rotary coupler in the tube extending to the collection bag.

4. The urinary catheter system of claim 3, the rotary union assembly including a cover over the rotary coupler.

5. The urinary catheter system of claim 1, including a leg strap connected to the rotary union assembly.

6. The urinary catheter assembly of claim 1, wherein the rotary union assembly is proximal to the condom-catheter.

7. The urinary catheter assembly of claim 1, wherein the rotary union assembly is proximal to the collection bag.

8. A rotary union assembly for use with a urinary catheter system comprising a condom-catheter and a collection bag, with a tube extending from proximal the condom-catheter to a junction on the tube, and extending from the junction to the collection bag, the junction in the tube between the condom-catheter and the collection bag, and including a rotary coupler sealed from leaking and having a female mating piece and a male mating piece that rotates freely within the female mating piece.

9. The junction of claim 8, including a cover over the rotary coupler.

10. The junction of claim 8, including a pressure relief valve is disposed adjacent the rotary coupler in the tube extending to the collection bag.

11. The junction of claim 10, including a cover over the rotary coupler and the pressure relief valve.
